(19) United States
(12) Patent Application Publication
(10) Pub. No.: US 2025/0145553 A1
(43) Pub. Date: May 8, 2025

(54) RECYCLED CONTENT MONOETHYLENE GLYCOL

(71) Applicant: Eastman Chemical Company, Kingsport, TN (US)

(72) Inventors: Daryl Bitting, Longview, TX (US); David Eugene Slivensky, Tatum, TX (US); Xianchun Wu, Longview, TX (US)

(73) Assignee: Eastman Chemical Company, Kingsport, TN (US)

(21) Appl. No.: 18/837,914

(22) PCT Filed: Feb. 14, 2023
(86) PCT No.: PCT/US2023/062545

§ 371 Date: Aug. 13, 2024

Related U.S. Application Data
(60) Provisional application No. 63/268,145, filed on Feb. 17, 2022.

(51) Int. Cl. C07C 29/149

(52) U.S. Cl. CPC C07C 29/149

(57) ABSTRACT

Recycled content monoethylene glycol (r-MEG) is produced using a process and system that applies physical and/or credit-based recycled content from one or more feed materials to the MEG produced from the feed materials. Recycled content diethylene glycol (DEG) can be formed in a similar manner.

### BACKGROUND

Monoethylene glycol (MEG) is an important chemical used in a wide variety of applications. For example, monoethylene glycol is a key ingredient in forming polymers such as polyethylene terephthalate, or PET. Monoethylene glycol-containing polymers have a wide variety of end uses, including as films, fibers, and resins for packaging, textile, and bottles. Additionally, monoethylene glycol is also used as a dehydrating agent, coolant, and anti-freeze, as well as a chemical intermediate for other valuable commercial and industrial chemicals.

The demand for recycled chemical products continues to grow, but there is no clear path to recycled monoethylene glycol through mechanical recycling. Thus, there exists a need for a commercial process to produce recycled content monoethylene glycol.

### SUMMARY

In one aspect, the present technology concerns a process for producing monoethylene glycol having recycled content (r-MEG), the process comprising: (a) hydrocarboxylating a first formaldehyde with a first carbon monoxide to form a glycolic acid; (b) esterifying the glycolic acid with a first methanol to form methyl glycolate; and (c) hydrogenating the methyl glycolate with a first hydrogen to form monoethylene glycol (MEG), wherein the MEG comprises recycled content from one or more of the following source materials—(i) waste plastic; (ii) recycled content carbon monoxide (r-CO); (iii) recycled content formaldehyde (r-formaldehyde); (iv) recycled content methanol (r-methanol); and (v) recycled content hydrogen (r-H2).

In one aspect, the present technology concerns a process for producing monoethylene glycol having recycled content (r-MEG), the process comprising: (a) hydroformylating a first formaldehyde with a first syngas to form glycol aldehyde; and (b) hydrogenating the glycol aldehyde with a first hydrogen to form monoethylene glycol (MEG), wherein the MEG comprises recycled content from one or more of the following source materials—(i) waste plastic; (ii) recycled content syngas (r-syngas); (iii) recycled content formaldehyde (r-formaldehyde); and (iv) recycled content hydrogen (r-H2).

### BRIEF DESCRIPTION OF THE DRAWINGS

FIG. 1a is a block flow diagram illustrating the main steps of a process and facility for making recycled content monoethylene glycol (r-MEG) via a glycolic acid intermediate and with methanol formed from syngas;

FIG. 1b is a block flow diagram illustrating the main steps of a process and facility for making recycled content monoethylene glycol (r-MEG) via a glycolic acid intermediate and with methanol formed from methane;

FIG. 1c is a block flow diagram illustrating the main steps of a process and facility for making recycled content monoethylene glycol (r-MEG) via a glycolaldehyde intermediate and with methanol formed from syngas;

FIG. 2 is a block flow diagram illustrating the main steps of a process and facility for making recycled content monoethylene glycol (r-MEG) as generally shown in FIG. 1a, where the r-MEG has credit-based recycled content from one or more source materials; and

FIG. 3 is a block flow diagram illustrating the main steps of a process and facility for making recycled content monoethylene glycol (r-MEG) as generally shown in FIG. 1a, where the r-MEG has both physical and credit-based recycled content from one or more source materials.

### DETAILED DESCRIPTION

We have discovered new methods and systems for producing monoethylene glycol (MEG) having recycled content. More specifically, we have discovered a process and facility for producing MEG where recycled content from waste materials, such as waste plastic, are applied to MEG in a manner that promotes the recycling of waste plastic and provides MEG with substantial amounts of recycled content.

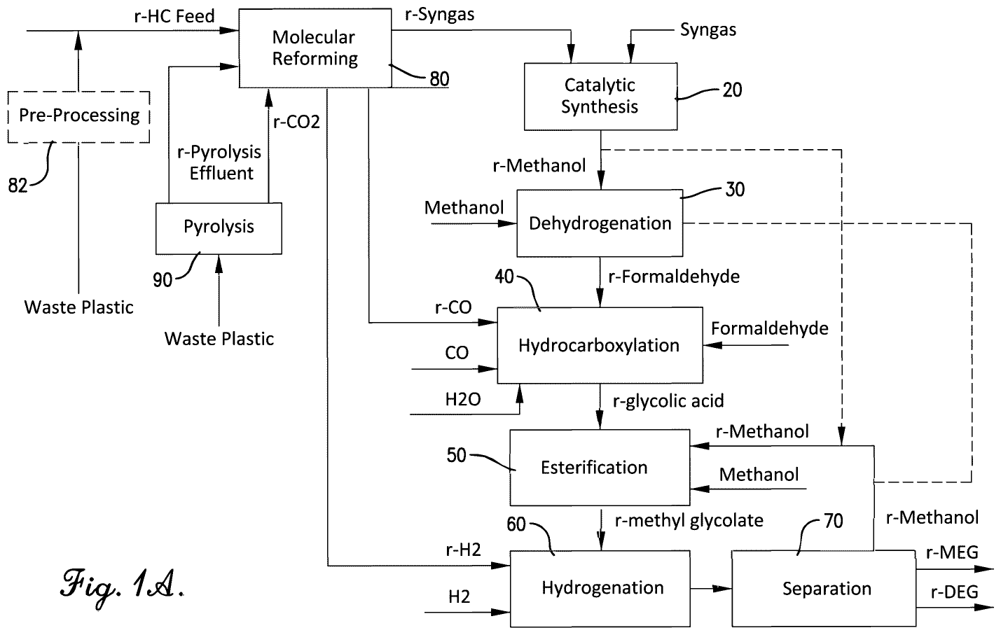

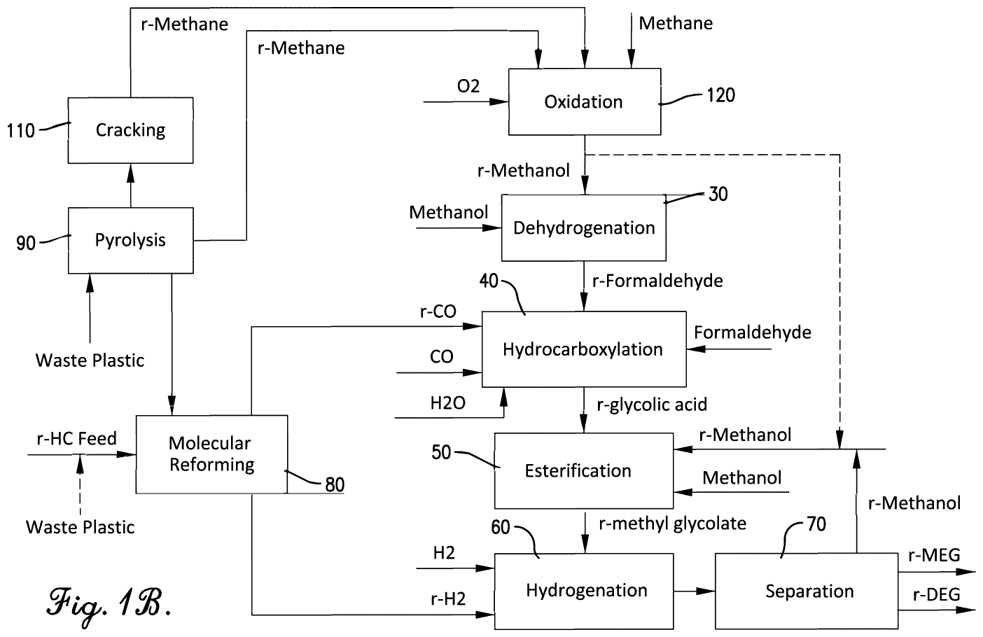

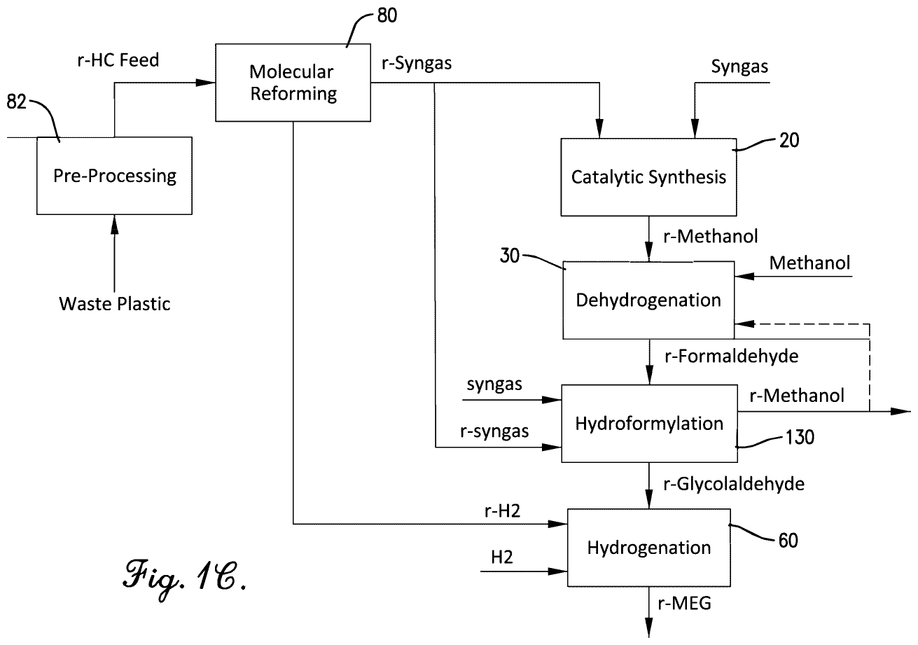

In general, there are two main chemical pathways for synthesizing monoethylene glycol. The first pathway includes the dehydration of methanol (alone or in the presence of an oxidizing agent) to form formaldehyde, which is then subjected to hydrocarboxylation in the presence of carbon monoxide (CO) to provide glycolic acid. The glycolic acid is then esterified with methanol to form methyl glycolate, which is then hydrogenated to form a mixture of monoethylene glycol and diethylene glycol (DEG). The MEG and DEG can be separated and recovered as individual products. In some cases (as shown generally in FIG. 1a), the methanol dehydrated to provide formaldehyde can be formed by catalytic synthesis of syngas, while in other cases (as shown generally in FIG. 1b), the methanol can be formed by oxidation of methane.

The second chemical pathway for producing MEG is generally depicted in FIG. 1c and also includes dehydrogenation of methanol to produce formaldehyde. According to this pathway, the formaldehyde is hydroformylated with syngas to produce glycolaldehyde. The glycolaldehyde is then hydrogenated to form MEG. As with the other chemical pathway, the methanol used to form the formaldehyde can itself be produced via catalytic synthesis of syngas and/or from oxidation of methane.

The resulting MEG can include recycled content from one or more source materials, including, for example, waste plastic, recycled content carbon monoxide (r-CO), recycled content formaldehyde (r-formaldehyde), recycled content methanol (r-methanol), and recycled content hydrogen (r-H2). The recycled content in the MEG can be physical and may originate directly from one or more of these streams and/or the recycled content may be credit-based and applied to a target stream in the MEG process from one or more of these source streams.

Turning now to FIG. 1a, one embodiment of a process and facility for forming MEG with physical (direct) recycled content is provided. The embodiment depicted in FIG. 1a illustrates a process/facility that produces MEG via a glycolic acid intermediate and utilizes methanol formed from syngas. The recycled content in the MEG formed in the facility shown in FIG. 1a can originate from the pyrolysis of waste plastic and/or from the molecular reforming of a recycled content hydrocarbon feed (r-HC Feed) originating all, or in part, from waste plastic.

The MEG process/facility shown in FIG. 1a includes a molecular reforming step/facility 80, a catalytic synthesis step/facility 20, a dehydrogenation step/facility 30, a hydrocarboxylation step/facility 40, an esterification step/facility 50, a hydrogenation step/facility 60, and a separation step/facility 70. In some embodiments, two or more of the molecular reforming facility 80, synthesis facility 20, dehydrogenation facility 30, hydrocarboxylation facility 40, esterification facility 50, hydrogenation facility 60, and separation facility 70 may be co-located. As used herein, the term “co-located” refers to the characteristic of at least two objects being situated on a common physical site, and/or within 1, within 0.75, within 0.5, or within 0.25 miles of each other, measured as a straight-line distance between two designated points.

In some embodiments, two or more of the molecular reforming facility 80, synthesis facility 20, dehydrogenation facility 30, hydrocarboxylation facility 40, esterification facility 50, hydrogenation facility 60, and separation facility 70 may be located remotely from one another. As used herein, the term “located remotely” refers to a distance of greater than 1, greater than 5, greater than 10, greater than 50, greater than 100, greater than 500, greater than 1000, or greater than 10,000 miles between two facilities, sites, or reactors.

When two or more of the above facilities are co-located, the facilities may be integrated in one or more ways. Examples of integration include, but are not limited to, heat integration, utility integration, waste-water integration, mass flow integration via conduits, office space, cafeterias, integration of plant management, IT department, maintenance department, and sharing of common equipment and parts, such as seals, gaskets, and the like.

In some embodiments, at least one of the two or more of the molecular reforming facility 80, synthesis facility 20, dehydrogenation facility 30, hydrocarboxylation facility 40, esterification facility 50, hydrogenation facility 60, and separation facility 70 can be a commercial scale facility/process receiving a feed stream at an average annual feed rate of at least 100, or at least 500, or at least 1,000, at least 2,000, at least 5,000, at least 10,000, at least 50,000, or at least 100,000 pounds per hour, averaged over one year. Further, the one or more of these facilities can produce a product stream (including a recycled content product stream) at an average annual rate of at least 100, or at least 1,000, or at least 5,000, at least 10,000, at least 50,000, or at least 75,000 pounds per hour, averaged over one year. When more than one product stream is produced, these rates can apply to the combined rate of all products.

Referring again to FIG. 1a, a hydrocarbon-containing feed stream can be introduced into the molecular reforming facility 80. As used herein, the term “molecular reforming” refers to conversion of a carbon-containing feed into syngas (CO, CO2, and H2). Molecular reforming encompasses both steam reforming and partial oxidation (POX) gasification. As used herein, the term “steam reforming” refers to the conversion of a carbon-containing feed into syngas via reaction with water. The steam reforming can be steam methane reforming and the carbon-containing feed can be a methane-containing stream, such as natural gas. As used herein, the term “partial oxidation (POX) gasification” or “POX gasification” refers to high temperature conversion of a carbon-containing feed into syngas, (carbon monoxide, hydrogen, and carbon dioxide), where the conversion is carried out in the presence of a less than stoichiometric amount of oxygen. The carbon containing-feed to POX gasification can include solids, liquids, and/or gases.

In some embodiments, at least a portion of the hydrocarbon-containing feed stream to the molecular reforming step/facility 80 can be a recycled content hydrocarbon feed (r-HC feed). The hydrocarbon-containing feed stream (or r-HC feed) introduced into the molecular reforming step/facility 80 can be comprises a solid-phase, a liquid-phase, or a gas-phase feed. Examples of suitable types of feed include methane, natural gas, naphtha, and coal (or coal slurry). In some cases, the r-HC feed may also include non-recycled content. When the r-HC feed includes recycled content, the resulting syngas formed in the molecular reforming facility comprises recycled content syngas (r-syngas). In some embodiments, the r-syngas has a recycled content of at least 5, at least 25, at least 50, at least 75, at least 90, or at least 99 percent, or it can have 100 percent recycled content.

The r-HC feed introduced into the molecular reforming step/facility 80 may include waste plastic or a stream including recycled content from waste plastic. When the r-HC feed includes waste plastic, it may be present in a solid form (e.g., as granules or a powder) or in a liquid form (e.g., dissolved in a solvent or combined with an aqueous liquid to form a slurry). Prior to entering the molecular reforming step/facility 80, the waste plastic may have passed through an optional pre-processing zone 82 as shown in FIG. 1a to process it into a desirable form. Examples of steps including in the pre-processing zone 82 can include, but are not limited to, grinding, chopping, dissolving, and slurry formation. Specific configurations of the pre-processing zone 82 depends on the type of waste plastic feed and the type of molecular reforming.

In some embodiments, as shown in FIG. 1a, at least a portion of the r-HC feed to the molecular reforming facility 80 can originate from an optional pyrolysis step/facility 90. When present, the pyrolysis step/facility 90 pyrolyzes waste plastic to provide a stream of recycled content pyrolysis effluent (r-pyrolysis effluent). Optionally (not shown), the r-pyrolysis effluent can be separated into individual streams of recycled content pyrolysis gas (r-pygas), recycled content pyrolysis oil (r-pyoil), and recycled content pyrolysis residue (r-pyrolysis residue), all or a portion of which can be introduced into the molecular reforming step/facility 80, alone or in combination with one another and/or with the r-HC feed.

As used herein, the term “r-pyrolysis residue” refers to a composition obtained from waste plastic pyrolysis that comprises predominantly pyrolysis char and pyrolysis heavy waxes. As used herein, the term “pyrolysis char” refers to a carbon-containing composition obtained from pyrolysis that is solid at 200° C. and 1 atm. As used herein, the term “pyrolysis heavy waxes” refers to C20+ hydrocarbons obtained from pyrolysis that are not pyrolysis char, pyrolysis gas, or pyrolysis oil. As used herein, the term “r-pygas” refers to a composition obtained from waste plastic pyrolysis that is gaseous at 25° C. at 1 atm. As used herein, the terms “r-pyoil” refers to a composition obtained from waste plastic pyrolysis that is liquid at 25° C. and 1 atm.

The pyrolysis reaction involves chemical and thermal decomposition of sorted waste plastic introduced into the reactor. Although all pyrolysis processes may be generally characterized by a reaction environment that is substantially free of oxygen, pyrolysis processes may be further defined by other parameters such as the pyrolysis reaction temperature within the reactor, the residence time in the pyrolysis reactor, the reactor type, the pressure within the pyrolysis reactor, and the presence or absence of pyrolysis catalysts.

In some embodiments, the peak pyrolysis temperature in the pyrolysis reactor can range from 325 to 800° C., or 350 to 600° C., or 375 to 500° C., or 390 to 450° C., or 400 to 500° C., and the residence time of the feedstock within the pyrolysis reactor 22 can range from 1 second to 1 hour, or 10 seconds to 30 minutes, or 30 seconds to 10 minutes. The pressure within the pyrolysis reactor 22 can be maintained at atmospheric pressure or within the range of 0.1 to 60, or 0.2 to 10, or 0.3 to 1.5 barg.

Additionally, or in the alternative, a stream of carbon dioxide (CO2) separated from the r-pyrolysis effluent within the pyrolysis step/facility 90 may also be introduced into the molecular reforming facility as shown in FIG. 1a. This stream, when present, can include recycled content carbon dioxide (r-CO2) and may be used as feed to the molecular reforming step/facility 80.

In some embodiments, at least a portion of the r-syngas from the molecular reforming step/facility 80 can be reacted in the presence of a catalyst to form recycled content methanol (r-methanol). Additionally, or in the alternative, a stream comprising non-recycled content syngas may also be used to form methanol. At least a portion of the r-methanol (or methanol) can then be subjected to dehydrogenation to form recycled content formaldehyde (or formaldehyde). The dehydrogenation reaction can be carried out with or without the addition of an oxygenating agent (e.g., air). At least a portion, or all, of the methanol can be non-recycled content methanol. In some embodiments, the syngas, methanol, and/or formaldehyde can include at least 50, at least 75, at least 90, or 100 percent recycled content, individually.

As shown in FIG. 1a, the r-formaldehyde formed by dehydrogenation can be subjected to hydrocarboxylation in a hydrocarboxylation step/facility 40 with recycled content carbon monoxide (r-CO). In some embodiments, the r-CO may be provided by a co-located or remotely located molecular reforming facility 80. In some embodiments, at least a portion of the formaldehyde and/or CO may include non-recycled content. The resulting recycled content glycolic acid (r-glycolic acid) may then be esterified with methanol to form r-methyl glycolate. In some embodiments, the methanol may include non-recycled content methanol. Alternatively, or in addition, at least a portion of the methanol can be r-methanol. When r-methanol is used, it can originate from several sources, co-located or remote to the esterification step/facility. For example, at least a portion of the r-methanol can originate from the catalytic synthesis of r-syngas or from separation of the resulting r-MEG product, as shown in FIG. 1a.

After esterification, the r-methyl glycolate can then be hydrogenated with recycled content hydrogen (r-H2) and/or non-recycled content hydrogen. When r-H2 is used, it may originate from a molecular reforming step/facility 80 as shown in FIG. 1a. The product stream resulting from hydrogenation in the hydrogenation step/facility 60 includes both recycled content monoethylene glycol (r-MEG) and recycled content diethylene glycol (r-DEG), which can be separated to form a predominantly r-DEG and a predominantly r-MEG product stream in a separation step/facility. A stream of r-methanol is also produced from the separation step/facility 70 that can be used in other steps/facilities of the process including the esterification step/facility 50 to form r-methyl glycolate and/or the dehydrogenation step/facility 30 to form r-formaldehyde.

Turning now to FIG. 1b, another embodiment of a process and facility for forming MEG with physical (direct) recycled content is provided. The embodiment shown in FIG. 1b illustrates a process/facility for producing MEG via a glycolic acid intermediate, but with methanol formed from methane. The recycled content of the MEG formed in the process/facility shown in FIG. 1b can originate from the pyrolysis of waste plastic (and optional cracking of pyrolysis effluent streams) and/or from molecular reforming of a recycled content hydrocarbon feed (r-HC feed) originating all, or in part, from waste plastic.

The process/facility depicted in FIG. 1b differs from that in FIG. 1a in that the r-methanol dehydrated to form r-formaldehyde in the process/facility shown in FIG. 1b is formed from the oxidation of methane. The methane may include recycled content methane (r-methane) and/or non-recycled content methane. The resulting r-methanol (or methanol) formed in the oxidation step/facility can then pass through the remaining steps/facilities as described previously with respect to FIG. 1a.

The r-methane can include recycled content derived from waste plastic. For example, in some embodiments, the r-methane can be separated out of a pyrolysis effluent stream (e.g., from an r-pygas stream) in a pyrolysis step/facility 90 that pyrolyzes waste plastic. In other embodiments, at least a portion of a pyrolysis effluent stream from the pyrolysis step/facility 90 (e.g., as r-pygas and/or r-pyoil) can be introduced into one or more locations in a cracking step/facility 110 and a stream of r-methane withdrawn from the cracking step/facility 110 may be introduced into the oxidation zone. Additionally, in some cases as shown in FIG. 1b, a portion of the r-pyrolysis effluent (e.g., as r-pygas, r-pyoil, and/or r-pyrolysis residue) can be introduced into the molecular reforming step/facility 80 and converted to r-syngas (or r-CO and r-H2) as discussed previously with respect to FIG. 1a.

Turning now to FIG. 1c, yet another embodiment of a process and facility for forming MEG is physical (direct) recycled content is provided. The embodiment shown in FIG. 1c illustrates a process/facility following another chemical reaction mechanism that forms MEG via a glycolaldehyde intermediate and using methanol formed from syngas, as described in FIG. 1a. Alternatively, a similar glycolaldehyde chemical pathway could be followed, but using methanol formed by oxidation of methane, as described in FIG. 1b.

In the chemical process/facility shown in FIG. 1c, r-formaldehyde formed by dehydrogenation of methane is introduced into a hydroformylation step/facility 130, where the formaldehyde is reacted with syngas to form r-glycolaldehyde. As shown in FIG. 1c, at least a portion of the syngas can be recycled content syngas (r-syngas) formed in a remotely located or co-located molecular reforming step/facility 80. Alternatively, or in addition, the syngas may include non-recycled content.

The r-glycolaldehyde withdrawn from the hydroformylation step/facility 130 can then be hydrogenated in a hydrogenation step/facility 60 to form recycled content MEG (r-MEG). As shown in FIG. 1c, at least a portion, or all, of the hydrogen may be recycled content hydrogen and can, for example, originate from a molecular reforming step/facility 80. In some embodiments, at least a portion, or all, of the hydrogen may be non-recycled content hydrogen.

The stream of r-MEG produced in any of the facilities/processes described herein can have a recycled content of at least 5, at least 10, at least 15, at least 20, at least 25, at least 30, at least 35, at least 40, at least 45, at least 50, at least 55, or at least 65 percent and/or 100 percent, or less than 99, less than 95, less than 90, less than 85, less than 80, less than 75, or less than 70 percent. When also formed, the stream of r-DEG can have a similar recycled content.

The amount of physical recycled content in the r-MEG can be determined by tracing the amount of recycled material along a chemical pathway starting with waste plastic and ending with the r-MEG. The chemical pathway includes all chemical reactions and other processing steps (e.g., separations) between the starting material (e.g., waste plastic) and the MEG. In FIG. 1a, the chemical pathway includes optional pyrolysis, molecular reforming, catalytic synthesis, dehydrogenation, hydrocarboxylation, esterification, hydrogenation, and separation, and in FIG. 1b, the chemical pathway includes optional pyrolysis and cracking, molecular reforming, oxidation, dehydrogenation, hydrocarboxylation, esterification, hydrogenation, and separation. The chemical pathway shown in FIG. 1c includes optional pyrolysis, molecular reforming, catalytic synthesis, dehydrogenation, hydroformylation, and hydrogenation.

In one or more embodiments, a conversion factor can be associated with each step along the chemical pathway. The conversion factors account for the amount of the recycled content diverted or lost at each step along the chemical pathway. For example, the conversion factors can account for the conversion, yield, and/or selectivity of the chemical reactions along the chemical pathway.

The amount of recycled content applied to the r-MEG can be determined using one of variety of methods for quantifying, tracking, and allocating recycled content among various materials in various processes. One suitable method, known as “mass balance,” quantifies, tracks, and allocates recycled content based on the mass of the recycled content in the process. In certain embodiments, the method of quantifying, tracking, and allocating recycled content is overseen by a certification entity that confirms the accuracy of the method and provides certification for the application of recycled content to the r-MEG.

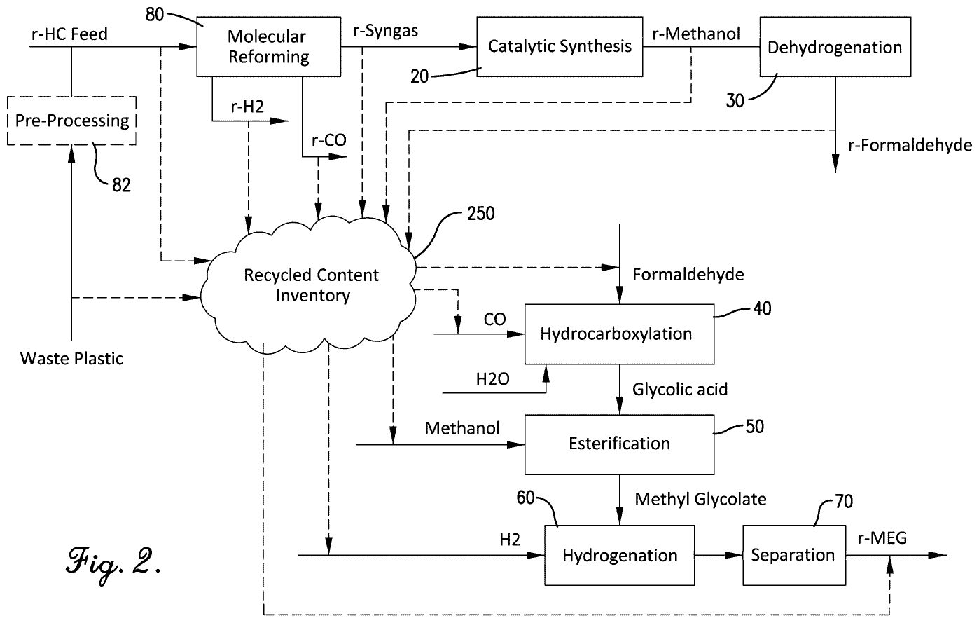

Turning now to FIG. 2, an embodiment where the r-MEG has no physical recycled content, but has credit-based recycled content, is provided. In the process and system depicted in FIG. 2, the r-syngas, r-CO, and r-H2 are not directly fed into the hydrocarboxylation or hydrogenation steps, nor is the r-methanol or r-formaldehyde directly fed into the hydrocarboxylation or esterification steps.

Instead, recycled content credits from the recycled content streams shown in FIG. 2 (e.g., the r-H2, r-CO, r-methanol, and/or r-formaldehyde) can be attributed to one or more other (and possibly, similar) streams in the process/facility. For example, the recycled content credits from one or more of the above streams can be attributed to the formaldehyde and/or CO fed into the hydrocarboxylation step/facility 40, the methanol fed into the esterification step/facility 50, or the hydrogen used in the hydrogenation step/facility 60. When the process/facility utilizes a reaction scheme as shown in FIG. 1b and/or 1c (not shown in FIG. 2), recycled content credits can be attributed to the methane fed to the oxidation step/facility 120 and/or to syngas fed to the hydroformylation step/facility 130, depending on the specific configuration. Alternatively, depending on the exact configuration of the process/facility, recycled content credits from the r-syngas could be attributed to a syngas fed into the catalytic synthesis step/facility 20, rather than being directly fed as shown in FIG. 2. In some cases, recycled content from the waste plastic and/or r-HC feed can be attributed to one or more of the streams, including the streams of MEG (and DEG, when provided).

As such, the r-H2, r-CO, r-methanol, r-formaldehyde (and, optionally, the r-syngas, r-methane, the r-HC feed and waste plastic) each act as a “source material” of recycled content credits and the formaldehyde and CO fed to the hydrocarboxylation, the methanol fed to esterification (or dehydration), the hydrogen fed to hydrogenation (and, optionally, the methane fed to oxidation and the syngas fed to hydroformylation) each act as a “target material” to which the recycled content credits are attributed.

In one or more embodiments, the source material has physical recycled content and the target material has less than 100 percent physical recycled content. For example, the source material can have at least 10, at least 25, at least 50, at least 75, at least 90, at least 99, or 100 percent physical recycled content and/or the target material can have less than 100, less than 99, less than 90, less than 75, less than 50, less than 25, less than 10, or less than 1 percent physical recycled content.

The ability to attribute recycled content credits from a source material to a target material removes the co-location requirement for the facility making the source material (with physical recycled content) and the facility making the MEG. This allows a chemical recycling facility/site in one location to process waste material into one or more recycled content source materials and then apply recycled content credits from those source materials to one or more target materials being processed in existing commercial facilities located remotely from the chemical recycling facility/site. Further, the use of recycled content credits allows different entities to produce the source material and the r-MEG. This allows efficient use of existing commercial assets to produce r-MEG. In one or more embodiments, the source material is made at a facility/site that is at least 0.1, at least 0.5, at least 1, at least 5, at least 10, at least 50, at least 100, at least 500, or at least 1000 miles from the facility/site where the target material is used to make MEG.

The attributing of recycled content credits from the source material (e.g., the r-H2 produced from pyrolysis of waste plastic and/or molecular reforming of r-HC feed) to the target material (e.g., the hydrogen fed to the hydrogenation step) can be accomplished by transferring recycled content credits directly from the source material to the target material. Alternatively, as shown in FIG. 2, recycled content credits can be applied from any of the waste plastic, the recycled content hydrocarbon feed to the molecular reforming step (r-HC feed), the r-syngas, the r-CO, the r-methanol, the r-H2, and/or the r-formaldehyde to the MEG (or DEG) via a recycled content inventory 250. The recycled content 250 can be a digital inventory or database used to record and track recycled content for various materials at various sites over various time periods.

When a recycled content inventory 250 is used, recycled content credits from the source material having physical recycled content (e.g., the waste plastic, the r-HC feed, the r-H2, the r-CO, the r-syngas, the r-methanol, the r-formaldehyde, and the r-methane, when present) are booked into the recycled content inventory 250. The recycled content inventory 250 can also contain recycled content credits from other sources and from other time periods. In one embodiment, recycled content credits in the recycled content inventory 250 can only be assigned to target materials having the same or similar composition as the source materials. For example, as shown in FIG. 2, recycled content credits booked into the recycled content inventory 250 from the r-H2 from molecular reforming can be assigned to the hydrogen fed to the hydrogenation step/facility 60 because the two streams have the same or similar compositions. However, in such embodiments, recycled content credits from r-H2 could not be assigned to the formaldehyde fed to the hydrocarboxylation step/facility 40 because the source and target materials would not be the same or similar.

In some embodiments, all or a portion of the recycled content credit can be applied to one or more target materials (e.g., H2 or methanol) upon receipt of one or more waste plastic containing materials at the facility. That is, the waste plastic (or recycled content hydrocarbon feed) need not be processed before applying the credit-based recycled content to the target material. Instead, receipt of the waste plastic (or waste-plastic containing material) at the facility can permit application of recycled content credit to one or more target materials. In most cases, however, such waste plastic will then be processed at the facility within 30, within 60, or within 90 days to produce one or more of the target materials.

Once recycled content credits have been attributed to the target material (e.g., the formaldehyde and/or CO fed into the hydrocarboxylation step/facility 40, the methanol fed into the esterification step/facility 50, the hydrogen fed into the hydrogenation step/facility 60, and/or the syngas fed into the hydroformylation step/facility 130), the amount of the credit-based recycled content allocated to the MEG is calculated by tracing the recycled content along the chemical pathway from the target material to the MEG. The chemical pathway includes all chemical reactions and other processing steps (e.g., separations) between the target material and the MEG, and a conversion factor can be associated with each step along the chemical pathway of the credit-based recycled content. The conversion factors account for the amount of the recycled content diverted or lost at each step along the chemical pathway. For example, the conversion factors can account for the conversion, yield, and/or selectivity of the chemical reactions along the chemical pathway.

As with the physical recycled content, the amount of credit-based recycled content applied to the r-MEG can be determined using one of variety of methods, such as mass balance, for quantifying, tracking, and allocating recycled content among various materials in various processes. In certain embodiments the method of quantifying, tracking, and allocating recycled content is overseen by a certification entity that confirms the accuracy of the method and provides certification for the application of recycled content to the r-MEG.

The r-MEG (or r-DEG if formed) produced by any of the processes shown in FIGS. 1a-c (or modifications thereof discussed herein) can have 25 to 90, 40 to 80, or 55 to 65 percent credit-based recycled content and less than 50, less than 25, less than 10, less than 5, or less than 1 percent physical recycled content. In certain embodiments, the r-MEG can have 10 to 80, 20 to 75, or 25 to 70 percent credit-based recycled content from one or more of the r-HC feed, r-H2, r-CO, r-methanol, and r-formaldehyde, individually.

In one or more embodiments, the recycled content of the r-MEG (or r-DEG, if formed) product can include both physical recycled content and credit-based recycled content. For example, the r-MEG can have at least 10, at least 20, at least 30, at least 40, or at least 50 percent physical recycled content and at least 10, at least 20, at least 30, at least 40, or at least 50 percent credit-based recycled content. As used herein, the term “total recycled content” refers to the cumulative amount of physical recycled content and credit-based recycled content from all sources.

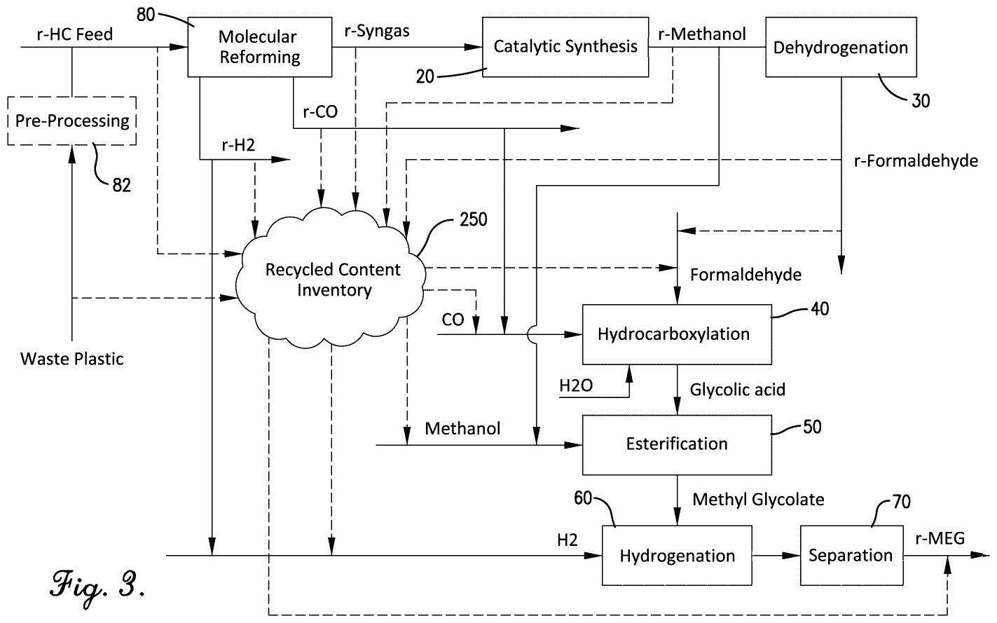

FIG. 3 illustrates several embodiments of an r-MEG production process and system, wherein physical recycled content and credit-based recycled content are attributed to the r-MEG. Any combination of physical (solid lines) and credit-based (dashed lines) recycled content shown in FIG. 3 can be used to form and/or can be attributed to MEG to thereby produce r-MEG. For example, physical recycled content can be supplied by at least 1, at least 2, at least 3, at least 4, or all of the sources shown in FIG. 3, including the r-H2, r-CO, r-syngas, r-methanol, and r-formaldehyde, while the credit-based recycled content can be supplied by one or more of the other sources shown in FIG. 3. In some embodiments, the r-MEG can include 10 to 60, 20 to 50, or 25 to 40 percent physical recycled content and 10 to 60, 20 to 50, or 25 to 40 percent credit-based recycled content. Alternatively, the r-MEG can include less than 15, less than 10, or less than 5 percent physical recycled content (or credit-based recycled content) and at least 85, at least 90, or at least 95 percent credit based recycled content (or physical recycled content).

For example, in some embodiments, physical recycled content can be provided by r-H2 to hydrogenation and r-CO to hydrocarboxylation, while credit-based recycled content can be provided by r-methanol and r-formaldehyde fed to the esterification step/facility 50 and the hydrocarboxylation step/facility 40, respectively. In other embodiments, the physical recycled content can be provided by the r-methanol and r-formaldehyde, while the credit-based recycled content can be provided by the r-CO and r-H2. Although not shown in FIG. 3, when the MEG is formed via an r-glycolaldehyde intermediate, physical recycled content can be supplied by the r-syngas and credit-based recycled content by the r-formaldehyde and/or r-methanol. Other permutations of this are shown in FIG. 3.

The recycled content MEG can be used in a variety of end use applications. For example, the r-MEG can be used as a starting material for a polyester or copolyesters resin used to form one or more of a wide variety of products including, but not limited to, bottles, containers, films for packaging including shrink films, and fibers for nonwoven and textile applications. It can also be used as an additive or dehydrating agent, as well as a coolant or antifreeze. It may also be used as a chemical intermediate and further reacted to form one or more additional valuable chemicals also having recycled content.

Although described herein with respect to MEG, it should be understood that all or a portion of the above-described facilities and processes can also be used to produce recycled content diethylene glycol (DEG).

In one embodiment or in combination with one or more embodiments disclosed herein, the pyrolysis reaction performed in the pyrolysis reactor can be carried out at a temperature of less than 700, less than 650, or less than 600° C. and at least 300, at least 350, or at least 400° C. The feed to the pyrolysis reactor can comprise, consists essentially of, or consists of waste plastic. The feed stream, and/or the waste plastic component of the feed stream, can have a number average molecular weight (Mn) of at least 3000, at least 4000, at least 5000, or at least 6000 g/mole. If the feed to the pyrolysis reactor contains a mixture of components, the Mn of the pyrolysis feed is the weighted average Mn of all feed components, based on the mass of the individual feed components.

The waste plastic in the feed to the pyrolysis reactor can include post-consumer waste plastic, post-industrial waste plastic, or combinations thereof. In certain embodiments, the feed to the pyrolysis reactor comprises less than 5, less than 2, less than 1, less than 0.5, or about 0.0 weight percent coal and/or biomass (e.g., lignocellulosic waste, switchgrass, fats and oils derived from animals, fats and oils derived from plants, etc.), based on the weight of solids in pyrolysis feed or based on the weight of the entire pyrolysis feed. The feed to the pyrolysis reaction can also comprise less than 5, less than 2, less than 1, or less than 0.5, or about 0.0 weight percent of a co-feed stream, including steam, sulfur-containing co-feed streams, and/or non-plastic hydrocarbons (e.g., non-plastic hydrocarbons having less than 50, less than 30, or less than 20 carbon atoms), based on the weight of the entire pyrolysis feed other than water or based on the weight of the entire pyrolysis feed.

Additionally, or alternatively, the pyrolysis reactor may comprise a film reactor, a screw extruder, a tubular reactor, a stirred tank reactor, a riser reactor, a fixed bed reactor, a fluidized bed reactor, a rotary kiln, a vacuum reactor, a microwave reactor, or an autoclave. The reactor may also utilize a feed gas and/or lift gas for facilitating the introduction of the feed into the pyrolysis reactor. The feed gas and/or lift gas can comprise nitrogen and can comprise less than 5, less than 2, less than 1, or less than 0.5, or about 0.0 weight percent of steam and/or sulfur-containing compounds.

In one embodiment or in combination with one or more embodiments disclosed herein, the cracker furnace can be operated at a product outlet temperature (e.g., coil outlet temperature) of at least 700, at least 750, at least 800, or at least 850° C. The feed to the cracker furnace can have a number average molecular weight (Mn) of less than 3000, less than 2000, less than 1000, or less than 500 g/mole. If the feed to the cracker furnace contains a mixture of components, the Mn of the cracker feed is the weighted average Mn of all feed components, based on the mass of the individual feed components. The feed to the cracker furnace can comprise less than 5, less than 2, less than 1, less than 0.5, or 0.0 weight percent of coal, biomass, and/or solids. In certain embodiments, a co-feed stream, such as steam or a sulfur-containing stream (for metal passivation) can be introduced into the cracker furnace. The cracker furnace can include both convection and radiant sections and can have a tubular reaction zone (e.g., coils in one or both of the convection and radiant sections). Typically, the residence time of the streams passing through the reaction zone (from the convection section inlet to the radiant section outlet) can be less than 20 seconds, less than 10 seconds, less than 5 seconds, or less than 2 seconds.

When a numerical sequence is indicated, it is to be understood that each number is modified the same as the first number or last number in the numerical sequence or in the sentence, e.g., each number is “at least,” or “up to” or “not more than” as the case may be; and each number is in an “or” relationship. For example, “at least 10, 20, 30, 40, 50, 75 wt. % . . . ” means the same as “at least 10 wt. %, or at least 20 wt. %, or at least 30 wt. %, or at least 40 wt. %, or at least 50 wt. %, or at least 75 wt. %,” etc.; and “not more than 90 wt. %, 85, 70, 60 . . . ” means the same as “not more than 90 wt. %, or not more than 85 wt. %, or not more than 70 wt. % . . . ” etc.; and “at least 1%, 2%, 3%, 4%, 5%, 6%, 7%, 8%, 9% or 10% by weight . . . ” means the same as “at least 1 wt. %, or at least 2 wt. %, or at least 3 wt. % . . . ” etc.; and “at least 5, 10, 15, 20 and/or not more than 99, 95, 90 weight percent” means the same as “at least 5 wt. %, or at least 10 wt. %, or at least 15 wt. % or at least 20 wt. % and/or not more than 99 wt. %, or not more than 95 wt. %, or not more than 90 weight percent . . . ” etc.

### Claim Supporting Description—First Embodiment

In a first embodiment of the present technology there is provided a process for producing monoethylene glycol having recycled content (r-MEG) and/or diethylene glycol having recycled content (r-DEG), the process comprising: (a) hydrocarboxylating a first formaldehyde with a first carbon monoxide to form a glycolic acid; (b) esterifying the glycolic acid with a first methanol to form methyl glycolate; and (c) hydrogenating the methyl glycolate with a first hydrogen to form monoethylene glycol (MEG) and/or diethylene glycol (DEG), wherein the MEG and/or DEG comprises recycled content from one or more of the following source materials—(i) waste plastic; (ii) recycled content carbon monoxide (r-CO); (iii) recycled content formaldehyde (r-formaldehyde); (iv) recycled content methanol (r-methanol); and (v) recycled content hydrogen (r-H2).

The first embodiment described in the preceding paragraph can also include one or more of the additional aspects listed below. The each of the following additional aspects of the first embodiment can be standalone features or can be combined with one or more of the other additional aspects to the extent consistent. Additionally, the following bullet pointed paragraphs can be viewed as dependent claim features having levels of dependency indicated by the degree of indention in the bulleted list (i.e., a feature indented further than the feature(s) listed above it is considered dependent on the feature(s) listed above it).
wherein the first methanol comprises non-recycled content.
wherein the first formaldehyde comprises non-recycled content.
wherein the first hydrogen comprises non-recycled content.
further comprising, separating the hydrogenated methyl glycolate to form a predominantly r-MEG stream and a predominantly recycled content diethylene glycol (r-DEG) stream.
   wherein the separating also produces a recycle methanol stream.
      wherein the first methanol comprises at least a portion of the recycle methanol stream.
      further comprising dehydrogenating at least a portion of the recycle methanol stream to provide the first formaldehyde.
further comprising converting a first syngas stream into a second methanol stream and dehydrogenating at least a portion of the second methanol to form the first formaldehyde.
   wherein the first methanol comprises at least a portion of the second methanol.
further comprising oxidizing a first methane with oxygen to form a second methanol and dehydrogenating at least a portion of the second methanol to form the first formaldehyde.
   wherein the first methanol comprises at least a portion of the second methanol.
further comprising forming recycled content syngas (r-syngas) by molecular reforming a hydrocarbon feed stream comprising recycled content from waste plastic.
   wherein at least one of the following criteria (i) through (iii) are met—
      (i) the first carbon monoxide comprises recycled content carbon monoxide (r-CO) produced during the making of the r-syngas;
      (ii) the first formaldehyde is formed from at least a portion of the r-syngas; and
      (iii) the first hydrogen comprises recycled content hydrogen (r-H2) produced during the making of the r-syngas.
   wherein at least a portion of the first formaldehyde is produced by converting a first syngas into methanol and dehydrogenating at least a portion of the methanol.
      wherein the first syngas comprises non-recycled content.
      wherein the first syngas comprises at least a portion of the r-syngas.
   wherein the molecular reforming is carried out in a partial oxidation reformer.
   wherein the molecular reforming is carried out in a steam reformer.
   wherein the hydrocarbon feed stream comprises recycled content from the pyrolysis of waste plastic.
      wherein the hydrocarbon feed stream comprises recycled content pyrolysis oil (r-pyoil), recycled content pyrolysis gas (r-pygas), recycled content pyrolysis residue (r-pyrolysis residue), and/or recycled content carbon dioxide (r-CO2).
   wherein the hydrocarbon feed stream comprises non-recycled content.
      wherein the non-recycled content comprises predominantly C2 to C5 hydrocarbons.
      wherein the non-recycled content comprises predominantly C5 to C22 hydrocarbons.
   wherein the hydrocarbon feed stream is a gas-phase, solid-phase, or liquid-phase stream.
   further comprising separating at least a portion of the r-syngas to form a recycled content hydrogen (r-H2), wherein the first hydrogen comprises at least a portion of the r-H2.
      wherein the first hydrogen comprises non-recycled content.
further comprising dehydrogenating a second methanol to thereby produce at least a portion of the first formaldehyde.
   wherein the second methanol comprises a recycled content methanol (r-methanol).
      wherein the second methanol comprises non-recycled content.
   further comprising oxidizing a first methane to thereby produce at least a portion of the second methanol.
      wherein the first methane is a recycled content methane (r-methane) and is formed by pyrolyzing waste plastic.
      wherein the first methane is a recycled content methane (r-methane) and is formed by cracking a hydrocarbon-containing stream formed by pyrolyzing waste plastic.
      wherein the first methane comprises non-recycled content.
wherein the MEG and/or DEG comprises physical recycled content from one or more of the source materials.
wherein the MEG and/or DEG comprises credit-based recycled content from one or more of the source materials.
wherein the MEG and/or DEG comprises both physical and credit-based recycled content from one or more of the source materials.
wherein hydrocarboxylating, esterifying, and hydrogenating are carried out in an MEG and/or DEG production facility, wherein at least one of the source materials providing recycled content to the MEG and/or DEG is produced in a remote source facility that is located at least 5, 10, 100, 500, 1000 or 10,000 miles from the MEG and/or DEG production facility.
   wherein the MEG and/or DEG comprises credit-based recycled content from the at least one source material produced in the remote source facility.
wherein hydrocarboxylating, esterifying, and hydrogenating are carried out in an MEG and/or DEG production facility, wherein at least one of the source materials providing recycled content to the MEG and/or DEG is produced in an on-site source facility that is located within 5, 1, 0.5, or 0.25 miles from the MEG and/or DEG production facility.
   wherein the MEG and/or DEG comprises credit-based recycled content from the at least one source material produced in the remote source facility.
wherein the MEG and/or DEG comprises recycled content from at least two (three, four, all) of the source materials.
wherein the MEG and/or DEG comprises recycled content from waste plastic.
   wherein the recycled content comprises credit-based recycled content.
   wherein the recycled content comprises physical recycled content.
wherein the first carbon monoxide comprises r-CO and the MEG and/or DEG comprises recycled content from the r-CO.
   wherein the recycled content comprises credit-based recycled content.
   wherein the recycled content comprises physical recycled content.
wherein the first formaldehyde comprises r-formaldehyde and the MEG and/or DEG comprises recycled content from the r-formaldehyde.
   wherein the recycled content comprises credit-based recycled content.
   wherein the recycled content comprises physical recycled content.
wherein the first hydrogen comprises r-H2 and the MEG and/or DEG comprises recycled content from the r-H2.
   wherein the recycled content comprises credit-based recycled content.
   wherein the recycled content comprises physical recycled content.
wherein the first methanol comprises r-methanol and the MEG and/or DEG comprises recycled content from the r-methanol.
   wherein the recycled content comprises credit-based recycled content.
   wherein the recycled content comprises physical recycled content.
further comprising apply credit-based recycled content to the MEG and/or DEG from the one or more source materials, wherein the applying includes (i) attributing recycled content from at least one of the source materials having physical recycled content to at least one target material via recycled content credits, (ii) tracing recycled content along at least one chemical pathway from the at least one target material to the MEG and/or DEG, and (iii) allocating recycled content to the MEG and/or DEG based at least in part on the tracing of recycled content along the chemical pathway.
   wherein attributing includes (i) booking recycled content credits attributable to the at least one source material into a digital inventory and (ii) assigning recycled content credits from the digital inventory to the target material, wherein the tracing includes determining one or more conversion factors for one or more chemical reactions along the chemical pathway, wherein the attributing includes assigning credit-based recycled content from a digital inventory to the target material, wherein the conversion factors determine how much of the credit-based recycled content applied to the target material is allocated to the MEG and/or DEG.
   wherein at least one of the following criteria (i) through (iv) is met—
      (i) the source material comprises the r-formaldehyde having physical recycled content and the target material comprises the first formaldehyde;
      (ii) the source material comprises the r-CO having physical recycled content and the target material comprises the first carbon monoxide;
      (iii) the source material comprises the r-H2 having physical recycled content and the target material comprises the first hydrogen; and
      (iv) the source material comprises the r-methanol having physical recycled content and the target material comprises the first methanol.
wherein the recycled content of the source material is from waste plastic.
wherein the source material and the target material have substantially the same physical composition.
wherein at least 50, 75, 90, 95, 99, or 100 weight percent of the source material is identical to the target material.
wherein the r-MEG and/or r-DEG has a total recycled content of at least 10, 20, 30, 40, 50, 75, 90, 95, or 100 percent.
wherein the r-MEG and/or r-DEG has at least 10, 20, 30, 40, 50 percent physical recycled content and at least 10, 20, 30, 40, or 50 percent credit-based recycled content.
wherein the source material has physical recycled content and the target material has less than 100 percent physical recycled content.
wherein the source material has at least 10, 25, 50, 75, 90, or 99 percent physical recycled content.
wherein the target material has less than 99, 90, 75, 50, 25, 10, or 1 percent physical recycled content.
wherein the source material has 100 percent physical recycled content and the target material has no physical recycled content.
wherein none of the first formaldehyde, first carbon monoxide, first methanol, and first hydrogen have physical recycled content.
wherein at least one (at least two, at least three, all) of the first formaldehyde, first carbon monoxide, first methanol, and first hydrogen have physical recycled content.
wherein the applying further comprises applying physical recycled content to at least a portion of the MEG and/or DEG so that the r-MEG and/or r-DEG has both physical recycled content and credit-based recycled content.
wherein the physical recycled content applied to the MEG and/or DEG is from at least one of the r-CO, r-H2, r-methanol, and r-formaldehyde.
wherein the target material comprises the formaldehyde and at least a portion of the credit-based recycled content allocated to the MEG and/or DEG is traced through a first chemical pathway from the formaldehyde to the MEG and/or DEG.
wherein the target material comprises the CO and at least a portion of the credit-based recycled content allocated to the MEG and/or DEG is traced through a first chemical pathway from the CO to the MEG and/or DEG.
wherein the target material comprises the methanol and at least a portion of the credit-based recycled content allocated to the MEG and/or DEG is traced through a first chemical pathway from the methanol to the MEG and/or DEG.
wherein the target material comprises the H2 and at least a portion of the credit-based recycled content allocated to the MEG and/or DEG is traced through a first chemical pathway from the H2 to the MEG and/or DEG.

### Claim Supporting Description—Second Embodiment

In a second embodiment of the present technology there is provided a process for producing monoethylene glycol having recycled content (r-MEG) and/or recycled content diethylene glycol (r-DEG), the process comprising: (a) hydroformylating a first formaldehyde with a first syngas to form glycol aldehyde; and (b) hydrogenating the glycol aldehyde with a first hydrogen to form monoethylene glycol (MEG), wherein the MEG and/or DEG comprises recycled content from one or more of the following source materials—(i) waste plastic; (ii) recycled content syngas (r-syngas); (iii) recycled content formaldehyde (r-formaldehyde); and (iv) recycled content hydrogen (r-H2).

The second embodiment described in the preceding paragraph can also include one or more of the additional aspects listed below. The each of the following additional aspects of the second embodiment can be standalone features or can be combined with one or more of the other additional aspects to the extent consistent.
wherein the first syngas comprises non-recycled content.
wherein the first formaldehyde comprises non-recycled content.
wherein the first hydrogen comprises non-recycled content.
further comprising forming recycled content syngas (r-syngas) by molecular reforming a hydrocarbon feed stream comprising recycled content from waste plastic.
   wherein at least one of the following criteria (i) through (iii) are met—
      (i) the first formaldehyde is formed from at least a portion of the r-syngas;
      (ii) the first syngas comprises at least a portion of the r-syngas; and
      (iii) the first hydrogen comprises recycled content hydrogen (r-H2) produced during the making of the r-syngas.
   wherein at least a portion of the first formaldehyde is produced by converting a second syngas into methanol and dehydrogenating at least a portion of the methanol.
      wherein the second syngas comprises at least a portion of the r-syngas.
      wherein the second syngas comprises non-recycled content.
   wherein the first syngas comprises at least a portion of the r-syngas.
      wherein the first syngas comprises non-recycled content.
   wherein the molecular reforming is carried out in a partial oxidation reformer.
   wherein the molecular reforming is carried out in a steam reformer.
   wherein the hydrocarbon feed stream comprises recycled content from the pyrolysis of waste plastic.
   wherein the hydrocarbon feed stream comprises non-recycled content.
      wherein the non-recycled content comprises predominantly C2 to C5 hydrocarbons.
      wherein the non-recycled content comprises predominantly C5 to C22 hydrocarbons.
   wherein the hydrocarbon feed stream is a gas-phase, solid-phase, or liquid-phase stream.
   wherein at least a portion of the recycled content in the hydrocarbon feed stream originates from pyrolysis of waste plastic.
   further comprising separating at least a portion of the r-syngas to form a recycled content hydrogen (r-H2), wherein the first hydrogen comprises at least a portion of the r-H2.
      wherein the first hydrogen comprises non-recycled content.
further comprising dehydrogenating a first methanol to thereby produce at least a portion of the first formaldehyde.
   wherein the first methanol comprises a recycled content methanol (r-methanol).
      wherein the first methanol comprises non-recycled content.
   wherein at least a portion of the first methanol is formed during the hydroformylation of step (a).
   further comprising converting a second syngas to thereby produce at least a portion of the first methanol.
      wherein the second syngas comprises recycled content syngas (r-syngas) formed by molecular reforming of a hydrocarbon feed stream comprising recycled content from waste plastic.
         wherein the molecular reforming is carried out in a partial oxidation reformer.
         wherein the molecular reforming is carried out in a steam reformer.
         wherein the hydrocarbon feed stream comprises recycled content from the pyrolysis of waste plastic.
   further comprising oxidizing a first methane to thereby produce at least a portion of the first methanol.
      wherein the first methane is a recycled content methane (r-methane) and is formed by pyrolyzing waste plastic.
      wherein the first methane is a recycled content methane (r-methane) and is formed by cracking a hydrocarbon-containing stream formed by pyrolyzing waste plastic.
      wherein the first methane comprises non-recycled content.
wherein the MEG and/or DEG comprises physical recycled content from one or more of the source materials.
wherein the MEG and/or DEG comprises credit-based recycled content from one or more of the source materials.
wherein the MEG and/or DEG comprises both physical and credit-based recycled content from one or more of the source materials.
wherein hydrocarboxylating, esterifying, and hydrogenating are carried out in an MEG and/or DEG production facility, wherein at least one of the source materials providing recycled content to the and/or DEG MEG is produced in a remote source facility that is located at least 5, 10, 100, 500, 1000 or 10,000 miles from the MEG and/or DEG production facility.
   wherein the MEG and/or DEG comprises credit-based recycled content from the at least one source material produced in the remote source facility.
wherein hydrocarboxylating, esterifying, and hydrogenating are carried out in an MEG and/or DEG production facility, wherein at least one of the source materials providing recycled content to the MEG and/or DEG is produced in an on-site source facility that is located within 5, 1, 0.5, or 0.25 miles from the MEG and/or DEG production facility.
   wherein the MEG and/or DEG comprises credit-based recycled content from the at least one source material produced in the remote source facility.
wherein the MEG and/or DEG comprises recycled content from at least two (three, four, all) of the source materials.
wherein the MEG and/or DEG comprises recycled content from waste plastic.
   wherein the recycled content comprises credit-based recycled content.
   wherein the recycled content comprises physical recycled content.
wherein the first carbon monoxide comprises r-CO and the MEG and/or DEG comprises recycled content from the r-CO.
   wherein the recycled content comprises credit-based recycled content.
   wherein the recycled content comprises physical recycled content.
wherein the first formaldehyde comprises r-formaldehyde and the MEG and/or DEG comprises recycled content from the r-formaldehyde.
   wherein the recycled content comprises credit-based recycled content.
   wherein the recycled content comprises physical recycled content.
wherein the first hydrogen comprises r-H2 and the MEG and/or DEG comprises recycled content from the r-H2.
   wherein the recycled content comprises credit-based recycled content.
   wherein the recycled content comprises physical recycled content.
wherein the first methanol comprises r-methanol and the MEG and/or DEG comprises recycled content from the r-methanol.
   wherein the recycled content comprises credit-based recycled content.
   wherein the recycled content comprises physical recycled content.
further comprising apply credit-based recycled content to the MEG and/or DEG from the one or more source materials, wherein the applying includes (i) attributing recycled content from at least one of the source materials having physical recycled content to at least one target material via recycled content credits, (ii) tracing recycled content along at least one chemical pathway from the at least one target material to the MEG and/or DEG, and (iii) allocating recycled content to the MEG and/or DEG based at least in part on the tracing of recycled content along the chemical pathway.
   wherein attributing includes (i) booking recycled content credits attributable to the at least one source material into a digital inventory and (ii) assigning recycled content credits from the digital inventory to the target material, wherein the tracing includes determining one or more conversion factors for one or more chemical reactions along the chemical pathway, wherein the attributing includes assigning credit-based recycled content from a digital inventory to the target material, wherein the conversion factors determine how much of the credit-based recycled content applied to the target material is allocated to the MEG and/or DEG.
wherein at least one of the following criteria (i) through (iv) is met—
   (i) the source material comprises the r-formaldehyde having physical recycled content and the target material comprises the first formaldehyde;
   (ii) the source material comprises the r-CO having physical recycled content and the target material comprises the first carbon monoxide;
   (iii) the source material comprises the r-H2 having physical recycled content and the target material comprises the first hydrogen; and
   (iv) the source material comprises the r-methanol having physical recycled content and the target material comprises the first methanol.
wherein the recycled content of the source material is from waste plastic.
wherein the source material and the target material have substantially the same physical composition.
wherein at least 50, 75, 90, 95, 99, or 100 weight percent of the source material is identical to the target material.
wherein the r-MEG and/or r-DEG has a total recycled content of at least 10, 20, 30, 40, 50, 75, 90, 95, or 100 percent.
wherein the r-MEG and/or r-DEG has at least 10, 20, 30, 40, 50 percent physical recycled content and at least 10, 20, 30, 40, or 50 percent credit-based recycled content.
wherein the source material has physical recycled content and the target material has less than 100 percent physical recycled content.
wherein the source material has at least 10, 25, 50, 75, 90, or 99 percent physical recycled content.
wherein the target material has less than 99, 90, 75, 50, 25, 10, or 1 percent physical recycled content.
wherein the source material has 100 percent physical recycled content and the target material has no physical recycled content.
wherein none of the r-syngas, r-H2, and r-formaldehyde have physical recycled content.
wherein at least one (at least two, all) of the r-syngas, r-H2, and r-formaldehyde have physical recycled content.
wherein the applying further comprises applying physical recycled content to at least a portion of the MEG and/or DEG so that the r-MEG and/or r-DEG has both physical recycled content and credit-based recycled content.
wherein the physical recycled content applied to the MEG and/or DEG is from at least one of the r-syngas, r-H2, and r-formaldehyde.
wherein the target material comprises the formaldehyde and at least a portion of the credit-based recycled content allocated to the MEG and/or DEG is traced through a first chemical pathway from the formaldehyde to the MEG and/or DEG.
wherein the target material comprises the syngas and at least a portion of the credit-based recycled content allocated to the MEG and/or DEG is traced through a first chemical pathway from the syngas to the MEG and/or DEG.
wherein the target material comprises the H2 and at least a portion of the credit-based recycled content allocated to the MEG and/or DEG is traced through a first chemical pathway from the H2 to the MEG and/or DEG.

### Definitions

It should be understood that the following is not intended to be an exclusive list of defined terms. Other definitions may be provided in the foregoing description, such as, for example, when accompanying the use of a defined term in context.

As used herein, the terms “a,” “an,” and “the” mean one or more.

As used herein, the term “and/or,” when used in a list of two or more items, means that any one of the listed items can be employed by itself or any combination of two or more of the listed items can be employed. For example, if a composition is described as containing components A, B, and/or C, the composition can contain A alone; B alone; C alone; A and B in combination; A and C in combination, B and C in combination; or A, B, and C in combination.

As used herein, the phrase “at least a portion” includes at least a portion and up to and including the entire amount or time period.

As used herein, the term “chemical pathway” refers to the chemical processing step or steps (e.g., chemical reactions, physical separations, etc.) between an input material and a product material, where the input material is used to make the product material.

As used herein, the term “chemical recycling” refers to a waste plastic recycling process that includes a step of chemically converting waste plastic polymers into lower molecular weight polymers, oligomers, monomers, and/or non-polymeric molecules (e.g., hydrogen, carbon monoxide, methane, ethane, propane, ethylene, and CO) that are useful by themselves and/or are useful as feedstocks to another chemical production process(es).

As used herein, the term “co-located” refers to the characteristic of at least two objects being situated on a common physical site, and/or within 5, 1, 0.5, or 0.25 miles of each other.

As used herein, the terms “comprising,” “comprises,” and “comprise” are open-ended transition terms used to transition from a subject recited before the term to one or more elements recited after the term, where the element or elements listed after the transition term are not necessarily the only elements that make up the subject.

As used herein, the terms “credit-based recycled content,” “non-physical recycled content,” and “indirect recycled content” all refer to matter that is not physically traceable back to a waste material, but to which a recycled content credit has been attributed.

As used herein, the term “directly derived” refers to having at least one physical component originating from waste material.

As used herein, the terms “including,” “include,” and “included” have the same open-ended meaning as “comprising,” “comprises,” and “comprise” provided above.

As used herein, the term “indirectly derived” refers to having an applied recycled content (i) that is attributable to waste material, but (ii) that is not based on having a physical component originating from waste material.

As used herein, the term “located remotely” refers to a distance of at least 0.1, 0.5, 1, 5, 10, 50, 100, 500, or 1000 miles between two facilities, sites, or reactors.

As used herein, the term “mass balance” refers to a method of tracking recycled content based on the mass of the recycled content in various materials.

As used herein, the terms “physical recycled content” and “direct recycled content” both refer to matter that is physically traceable back to a waste material.

As used herein, the term “predominantly” means more than 50 percent by weight. For example, a predominantly propane stream, composition, feedstock, or product is a stream, composition, feedstock, or product that contains more than 50 weight percent propane.

As used herein, the term “recycled content” refers to being or comprising a composition that is directly and/or indirectly derived from recycle material. Recycled content is used generically to refer to both physical recycled content and credit-based recycled content. Recycled content is also used as an adjective to describe material having physical recycled content and/or credit-based recycled content.

As used herein, the term “recycled content credit” refers to a non-physical measure of physical recycled content that can be directly or indirectly (i.e., via a digital inventory) attributed from a first material having physical recycled content to a second material having less than 100 percent physical recycled content.

As used herein, the term “total recycled content” refers to the cumulative amount of physical recycled content and credit-based recycled content from all sources.

As used herein, the term “waste material” refers to used, scrap, and/or discarded material.

As used herein, the terms “waste plastic” and “plastic waste” refer to used, scrap, and/or discarded plastic materials.

### Claims Not Limited to Disclosed Embodiments

The preferred forms of the invention described above are to be used as illustration only and should not be used in a limiting sense to interpret the scope of the present invention. Modifications to the exemplary embodiments, set forth above, could be readily made by those skilled in the art without departing from the spirit of the present invention.

The inventors hereby state their intent to rely on the Doctrine of Equivalents to determine and assess the reasonably fair scope of the present invention as it pertains to any apparatus not materially departing from but outside the literal scope of the invention as set forth in the following claims.

1. A process for producing monoethylene glycol having recycled content (r-MEG), the process comprising:
(a) hydrocarboxylating a first formaldehyde with a first carbon monoxide to form a glycolic acid;
(b) esterifying the glycolic acid with a first methanol to form methyl glycolate; and
(c) hydrogenating the methyl glycolate with a first hydrogen to form monoethylene glycol (MEG),
wherein the MEG comprises recycled content from one or more of the following source materials—(i) waste plastic; (ii) recycled content carbon monoxide (r-CO); (iii) recycled content formaldehyde (r-formaldehyde); (iv) recycled content methanol (r-methanol); and (v) recycled content hydrogen (r-H2).

2. The process of claim 1, further comprising forming recycled content syngas (r-syngas) by molecular reforming a hydrocarbon feed stream comprising recycled content from waste plastic and wherein at least one of the following criteria (i) through (iii) are met—
(i) the first carbon monoxide comprises recycled content carbon monoxide (r-CO) produced during the making of the r-syngas;
(ii) the first formaldehyde is formed from at least a portion of the r-syngas; and
(iii) the first hydrogen comprises recycled content hydrogen (r-H2) produced during the making of the r-syngas.

3. The process of claim 1, further comprising dehydrogenating a second methanol to thereby produce at least a portion of the first formaldehyde, and wherein the second methanol comprises a recycled content methanol (r-methanol).

4. The process of claim 3, further comprising oxidizing a first methane to thereby produce at least a portion of the second methanol, and wherein said first methane comprises recycled content methane (r-methane).

5. The process of claim 4, wherein at least a portion of the r-methane comes from the pyrolysis of waste plastic to form an r-pyrolysis effluent and separation of at least a portion of the r-pyrolysis effluent to form the r-methane.

6. The process of claim 3, further comprising converting syngas to thereby produce at least a portion of the second methanol, wherein said syngas comprises recycled content syngas (r-syngas).

7. The process of claim 1, further comprising, separating the hydrogenated methyl glycolate to form a predominantly MEG stream and a predominantly diethylene glycol (DEG) stream.

8. The process of claim 7, wherein the separating also produces a recycle methanol stream.

9. The process of claim 8, wherein the first methanol comprises at least a portion of the recycle methanol stream and/or further comprising dehydrogenating at least a portion of the recycle methanol stream to provide the first formaldehyde.

10. The process of claim 1, wherein the MEG comprises recycled content from at least two of the source materials.

11. The process of claim 1, wherein hydrocarboxylating, esterifying, and hydrogenating are carried out in an MEG production facility, wherein at least one of the source materials providing recycled content to the MEG is produced in a remote source facility that is located at least 5 miles from the MEG production facility.

12. The process of claim 1, further comprising apply credit-based recycled content to the MEG from the one or more source materials, wherein the applying includes (i) attributing recycled content from at least one of the source materials having physical recycled content to at least one target material via recycled content credits, (ii) tracing recycled content along at least one chemical pathway from the at least one target material to the MEG, and (iii) allocating recycled content to the MEG based at least in part on the tracing of recycled content along the chemical pathway.

13. The process of claim 1, wherein the MEG comprises physical recycled content from one or more of the source materials.

14. The process of claim 1, wherein the MEG comprises credit-based recycled content from one or more of the source materials.

15. A process for producing monoethylene glycol having recycled content (r-MEG), the process comprising:
(a) hydroformylating a first formaldehyde with a first syngas to form glycol aldehyde; and
(b) hydrogenating the glycolaldehyde with a first hydrogen to form monoethylene glycol (MEG),
wherein the MEG comprises recycled content from one or more of the following source materials—(i) waste plastic; (ii) recycled content syngas (r-syngas); (iii) recycled content formaldehyde (r-formaldehyde); and (iv) recycled content hydrogen (r-H2).

16. The process of claim 15, further comprising forming recycled content syngas (r-syngas) by molecular reforming a hydrocarbon feed stream comprising recycled content from waste plastic, and wherein at least one of the following criteria (i) through (iii) are met—
(i) the first syngas is recycled content syngas (r-syngas);
(ii) the first formaldehyde is formed from at least a portion of the r-syngas; and
(iii) the first hydrogen comprises recycled content hydrogen (r-H2) produced during the making of the r-syngas.

17. The process of claim 15, further comprising dehydrogenating a first methanol to thereby produce at least a portion of the first formaldehyde, and wherein at least one of the following (i) through (iii) is true: (i) wherein at least a portion of the first methanol is formed during the hydroformylation of step (a); (ii) further comprising converting a second syngas to thereby produce at least a portion of the first methanol; and (iii) further comprising oxidizing a first methane to thereby produce at least a portion of the first methanol.

18. The process of claim 15, further comprising apply credit-based recycled content to the MEG from the one or more source materials, wherein the applying includes (i) attributing recycled content from at least one of the source materials having physical recycled content to at least one target material via recycled content credits, (ii) tracing recycled content along at least one chemical pathway from the at least one target material to the MEG, and (iii) allocating recycled content to the MEG based at least in part on the tracing of recycled content along the chemical pathway.

19. The process of claim 15, wherein at least one of the following criteria (i) through (iii) is met—
(i) the source material comprises the r-formaldehyde having physical recycled content and the target material comprises the first formaldehyde;
(ii) the source material comprises the r-syngas having physical recycled content and the target material comprises the first syngas; and
(iii) the source material comprises the r-H2 having physical recycled content and the target material comprises the first hydrogen.

20. The process of claim 1, wherein the MEG comprises physical recycled content from one or more of the source materials.
